(19) United States
(12) Patent Application Publication
(10) Pub. No.: US 2020/0113806 A1
(43) Pub. Date: Apr. 16, 2020

(54) COSMETIC AGENT FOR TEMPORARILY SHAPING KERATIN FIBRES WITH POLYMER COMBINATION I

(71) Applicant: Henkel AG & Co. KGaA, Duesseldorf (DE)

(72) Inventors: JULIA BIBIANE LANGE, Bad Bramstedt (DE); DIANE METTEN, Hamburg (DE); CYRIELLE MARTINEZ, Hamburg (DE)

(73) Assignee: Henkel AG & Co. KGaA, Duesseldorf (DE)

(21) Appl. No.: 16/597,392

(22) Filed: Oct. 9, 2019

(30) Foreign Application Priority Data
Oct. 11, 2018 (DE) 10 2018 217 402.4

(51) Int. Cl. A61K 8/81; A61Q 5/06; A61K 8/73; A61K 8/31; A45D 7/04

(52) U.S. Cl. CPC A61K 8/8152; A61Q 5/06; A61K 8/737; A61K 8/31; A61K 8/736; A61K 8/8176; A61K 2800/5922; A61K 8/8182; A45D 7/04; A61K 2800/594; A61K 2800/5426; A61K 2800/22; A61K 2800/87; A61K 8/8158

(57) ABSTRACT

The application describes a cosmetic agent for temporarily shaping keratin fibres. The cosmetic agent includes, in a cosmetically acceptable carrier, a) at least one copolymer A formed from at least one monomer A1 selected from acrylic acid and/or methacrylic acid and at least one monomer A2 selected from acrylates and/or methacrylates, b) at least one cationic polymer B different from copolymer A and selected from cationic guar derivatives, c) at least one polymer C different from copolymer A and polymer B and the at least one polymer C is selected from homopolymers or copolymers of vinylpyrrolidone, and d) at least one propellant.

### CROSS-REFERENCE TO RELATED APPLICATION

This application claims priority to German Patent Application No. 10 2018 217 402.4, filed Oct. 11, 2018, which is incorporated herein by reference in its entirety.

### TECHNICAL FIELD

The application relates to cosmetic agents and products for temporarily shaping keratin fibres with an acrylate copolymer and keratin guar compound, and also to the use thereof. The application also describes a method with use of the cosmetic agent.

### BACKGROUND

The temporary shaping of hairstyles for a relatively long period of time up to several days generally requires the application of firming active ingredients. Appropriate agents for temporary shaping usually contain synthetic polymers and/or wax as firming active ingredient. Agents for assisting the temporary shaping of keratin fibres can be provided for example in the form of hairspray, hair wax, hair gel or hair mousse.

The most important property of an agent for temporarily shaping hair, also referred to hereinafter as styling agents, lies in providing the treated fibres with the strongest possible hold in the newly modelled form—i.e. a form impressed on the hair. Reference is also made to a strong hairstyle hold or to a high holding power of the styling agent. The hold of a hairstyle is determined fundamentally by the type and quantity of the used firming active ingredient, however the further constituents of the styling agent can also have an effect.

In addition to a high holding power, styling agents must also satisfy a wide range of further requirements. These can be divided roughly into properties on the hair, properties of the particular formulation, for example properties of the sprayed aerosol, and properties that concern the handling of the styling agent, wherein the properties on the hair are attributed particular importance. In particular, moisture resistance, low stickiness (tack), and a balanced conditioning effect can be cited. Furthermore, a styling agent should be universally usable for all hair types where possible and should be mild on the hair and skin.

In order to satisfy the different requirements, a multiplicity of synthetic polymers which are used in styling agents have already been developed as firming active ingredients.

Document WO 2009/059813 A2 discloses agents for temporarily shaping keratin fibres containing a specific combination of polymers. The polymer combination can comprise, for example, an acrylate copolymer and a cationic guar compound.

Although suitable polymers and polymer combinations have been developed for some time now for use in the field of temporary hair shaping, the previously attained results still have room for improvements, in particular with regard to the storage stability, ease of application, and the holding power of these agents. In particular, currently obtainable styling agents can still be improved insofar as a good combination of holding power and long-term hold (High Humidity Curl Retention) is not always sufficiently ensured.

From ecological viewpoints, it is additionally desirable to reduce the content of purely synthetic polymers in agents for temporarily shaping keratin fibres.

### BRIEF SUMMARY

One object of the present disclosure was to provide further suitable active ingredient combinations which are exemplified by good film-forming and/or firming properties and have a very high holding power, without detriment to the flexibility and good moisture resistance—in particular resistance to perspiration and water.

This object is achieved by a cosmetic agent for temporarily shaping keratin fibres, containing in a cosmetically acceptable carrier

a) at least one copolymer A formed from
a1) at least one monomer A1, selected from acrylic acid and/or methacrylic acid, and
a2) at least one monomer A2, selected from acrylates and/or methacrylates,
   b) at least one cationic polymer B different from copolymer A and selected from cationic guar derivatives,
   c) at least one polymer C different from copolymer A and polymer B and selected from homopolymers or copolymers of vinylpyrrolidone, and
   d) at least one propellant.

The (co)polymers A to B are known per se. The combination of the three components a) to c) leads to a significant increase in the moisture resistance and holding power of the agent, without detriment to the ease with which the agent can be washed out.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The term ‘keratin fibres’ is understood to mean, in principle, all animal hair, for example wool, horsehair, angora hair, fur, feathers, and products or textiles produced therefrom. Keratin fibres, however, are preferably human hair, in particular hair on the head and/or beard hair.

As first constituent essential to the present disclosure the agent contains at least one copolymer A. This copolymer has at least one monomer component A1 selected from acrylic acid and/or methacrylic acid, and at least one monomer component A2 selected from acrylates and/or methacrylates. The copolymer A may additionally comprise further structural units which are polymerised in by the addition of corresponding monomers at the time of polymerisation.

Monomer A1 is particularly preferably selected from acrylic acid and/or methacrylic acid, and monomer A2 is particularly preferably selected from methyl acrylate, methyl methacrylate, ethyl acrylate, ethyl methacrylate, propyl acrylate, propyl methacrylate, isopropyl acrylate, isopropyl methacrylate, octyl acrylate, octyl methacrylate, decyl acrylate, decyl methacrylate, lauryl acrylate, lauryl methacrylate, myristyl acrylate, myristyl methacrylate, cetyl acrylate, cetyl methacrylate, stearyl acrylate, stearyl methacrylate, eicosyl acrylate and eicosyl methacrylate, very particularly preferably from acrylic acid, methacrylic acid, methyl acrylate, methyl methacrylate, ethyl acrylate, ethyl methacrylate, lauryl acrylate, lauryl methacrylate, stearyl acrylate and stearyl methacrylate.

Particularly preferred copolymers A are:
copolymers of acrylic acid with methyl acrylate,
copolymers of acrylic acid with methyl methacrylate
copolymers of acrylic acid with ethyl acrylate,
copolymers of acrylic acid with ethyl methacrylate,
copolymers of acrylic acid with propyl acrylate,
copolymers of acrylic acid with propyl methacrylate,
copolymers of acrylic acid with isopropyl acrylate,
copolymers of acrylic acid with isopropyl methacrylate,
copolymers of acrylic acid with octyl acrylate,
copolymers of acrylic acid with octyl methacrylate,
copolymers of acrylic acid with decyl acrylate,
copolymers of acrylic acid with decyl methacrylate,
copolymers of acrylic acid with lauryl acrylate,
copolymers of acrylic acid with lauryl methacrylate,
copolymers of acrylic acid with myristyl acrylate,
copolymers of acrylic acid with myristyl methacrylate,
copolymers of acrylic acid with cetyl acrylate,
copolymers of acrylic acid with cetyl methacrylate,
copolymers of acrylic acid with stearyl acrylate,
copolymers of acrylic acid with stearyl methacrylate,
copolymers of acrylic acid with eicosyl acrylate,
copolymers of acrylic acid with eicosyl methacrylate,
copolymers of methacrylic acid with methyl acrylate,
copolymers of methacrylic acid with methyl methacrylate,
copolymers of methacrylic acid with ethyl acrylate,
copolymers of methacrylic acid with ethyl methacrylate,
copolymers of methacrylic acid with propyl acrylate,
copolymers of methacrylic acid with propyl methacrylate,
copolymers of methacrylic acid with isopropyl acrylate,
copolymers of methacrylic acid with isopropyl methacrylate,
copolymers of methacrylic acid with octyl acrylate,
copolymers of methacrylic acid with octyl methacrylate,
copolymers of methacrylic acid with decyl acrylate,
copolymers of methacrylic acid with decyl methacrylate,
copolymers of methacrylic acid with lauryl acrylate,
copolymers of methacrylic acid with lauryl methacrylate,
copolymers of methacrylic acid with myristyl acrylate,
copolymers of methacrylic acid with myristyl methacrylate,
copolymers of methacrylic acid with cetyl acrylate,
copolymers of methacrylic acid with cetyl methacrylate,
copolymers of methacrylic acid with stearyl acrylate,
copolymers of methacrylic acid with stearyl methacrylate,
copolymers of methacrylic acid with eicosyl acrylate,
copolymers of methacrylic acid with eicosyl methacrylate,
copolymers of acrylic acid and methacrylic acid with methyl acrylate,
copolymers of acrylic acid and methacrylic acid with methyl methacrylate
copolymers of acrylic acid and methacrylic acid with ethyl acrylate,
copolymers of acrylic acid and methacrylic acid with ethyl methacrylate,
copolymers of acrylic acid and methacrylic acid with propyl acrylate,
copolymers of acrylic acid and methacrylic acid with propyl methacrylate,
copolymers of acrylic acid and methacrylic acid with isopropyl acrylate,
copolymers of acrylic acid and methacrylic acid with isopropyl methacrylate,
copolymers of acrylic acid and methacrylic acid with octyl acrylate,
copolymers of acrylic acid and methacrylic acid with octyl methacrylate,
copolymers of acrylic acid and methacrylic acid with decyl acrylate,
copolymers of acrylic acid and methacrylic acid with decyl methacrylate,
copolymers of acrylic acid and methacrylic acid with lauryl acrylate,
copolymers of acrylic acid and methacrylic acid with lauryl methacrylate,
copolymers of acrylic acid and methacrylic acid with myristyl acrylate,
copolymers of acrylic acid and methacrylic acid with myristyl methacrylate,
copolymers of acrylic acid and methacrylic acid with cetyl acrylate,
copolymers of acrylic acid and methacrylic acid with cetyl methacrylate,
copolymers of acrylic acid and methacrylic acid with stearyl acrylate,
copolymers of acrylic acid and methacrylic acid with stearyl methacrylate,
copolymers of acrylic acid and methacrylic acid with eicosyl acrylate,
copolymers of acrylic acid and methacrylic acid with eicosyl methacrylate.

Particularly preferred agents contain as copolymer A a copolymer of methacrylic acid (MAA) and ethyl acrylate (EA).

It is preferred that the total amount of copolymers A, in relation to the weight of the easy-to-use agent, is from about 0.05 to about 5% by weight, preferably from about 0.1 to about 2% by weight and in particular from about 0.25 to about 1.5% by weight.

A second essential ingredient of the agent for temporarily shaping keratin fibres is at least one further polymer B from the group of cationic polymers, i.e. polymers which contain at least one monomer unit with a positively charged group. This cationic polymer is selected from cationic guar derivatives.

Here, agents which contain as cationic polymer B at least one guar 2-hydroxy-3-(trimethylammonio)propyl ether (INCI name: GUAR HYDROXYPROPYLTRIMONIUM CHLORIDE).

Other quaternised guar derivatives can also be used with preference. A further preferred embodiment of the present disclosure is constituted by agents that contain as cationic polymer at least one hydroxypropyl guar 2-hydroxy-3-trimethyl(ammonio)propyl ether (INCI name: HYDROXYPROPYL GUAR HYDROXYPROPYLTRIMONIUM CHLORIDE).

The cationic guar derivatives are preferably used within certain amount ranges. Here, agents that contain, in relation to the weight of the ready-to-use agent, from about 0.15 to about 15% by weight, preferably from about 0.3 to about 6% by weight and in particular from about 0.75 to about 4.5% by weight of polymer(s) B are preferred.

Agents in which the weight ratio of copolymer(s) A to polymer(s) B is from about 1:1 to about 1:10, more preferably from about 1:1 to about 1:5 and very particularly preferably from about 1:2 to about 1:4, are preferred.

A third essential ingredient of the agent for temporarily shaping keratin fibres is at least one polymer C different from copolymer A and polymer B and selected from homopolymers or copolymers of vinylpyrrolidone. The polymer C particularly preferably comprises a polymer selected from the group including polyvinylpyrrolidone (PVP), vinylpyrrolidone-vinyl acetate copolymer (INCI: VP/VA Copolymer), vinyl caprolactam/VP/dimethyl aminoethyl methacrylate copolymer (INCI), VP/DMAPA Acrylates Copolymer (INCI), Polyquaternium-11 (INCI), Polyquaternium-16 (INCI) and mixtures thereof.

Vinylpyrrolidone homopolymers (PVP) are sold for example under the name Luviskol® (BASF). The product range Luviskol® K (BASF), in which vinylpyrrolidone homopolymers are offered in different molecular weights, is obtainable in particular for use in cosmetic agents and styling products.

Vinylpyrrolidone/vinyl acetate copolymers are sold for example under the name Luviskol® VA (BASF) with different ratios of vinylpyrrolidone (VP) to vinyl acetate (VA). The water solubility increases with increasing VP content.

A vinylcaprolactam-vinylpyrrolidone-dimethylaminoethylmethylacrylate copolymer (INCI: Vinyl Caprolactam/VP/Dimethylaminoethyl Methacrylate Copolymer) is obtainable for example under the trade names Gaffix™ VC-713, Advantage S, Advantage LC-E, Advantage LC-A or Advantage HC-37 from ISP.

As vinylpyrrolidone/vinylcaprolactam/dimethylaminopropylacrylamide copolymer, the copolymer with the INCI name VP/Vinyl Caprolactam/DMAPA Acrylates Copolymer obtainable from ISP under the name Aquallex SF 40 is preferably used.

An VP/DMAPA Acrylates copolymer is sold for example under the name Styleze CC-10 from Ashland.

Polyquaternium-II is a quaternary ammoniumpolymer, which is formed by reacting diethyl sulfate with the copolymer of vinylpyrrolidone and dimethylaminoethyl methacrylate. Polyquaternium-11 obtainable for example as Luviquat® PQ 11 PN (BASF), Gafquat® 734, Gafquat® 755 or Gafquat® 755N (GAF).

Polyquaternium-16 is a polymer quaternary ammonium salt formed from methyl vinylimidazolium chloride and vinylpyrrolidone and is obtainable for example as Luviquat® FC 370, Luviquat® Style, Luviquat® FC 550 or Luviquat® Excellence (all BASF).

The vinylpyrrolidone-containing polymer C is preferably used within certain amount ranges. Here, agents that contain, in relation to the weight of the ready-to-use agent, from about 0.1 to about 20% by weight, preferably from about 0.5 to about 15% by weight and in particular from about 1 to about 10% by weight of polymer(s) C are preferred.

Particularly preferred agents contain as copolymer A a copolymer of methacrylic acid (MAA) and ethyl acrylate (EA), as polymer B Guar Hydroxypropyltrimonium Chloride (INCI) and as polymer C polyvinylpyrrolidone (PVP).

Particularly preferred agents contain as copolymer A a copolymer of methacrylic acid (MAA) and ethyl acrylate (EA), as polymer B Guar Hydroxypropyltrimonium Chloride and as polymer C vinylpyrrolidone-vinyl acetate copolymer (INCI: VP/VA Copolymer).

Agents that are also particularly preferred contain as copolymer A a copolymer of methacrylic acid (MAA) and ethyl acrylate (EA), as polymer B Guar Hydroxypropyltrimonium Chloride and as polymer C Vinyl Caprolactam/VP/Dimethylaminoethyl Methacrylate Copolymer (INCI).

Agents that are likewise particularly preferred contain as copolymer A a copolymer of methacrylic acid (MAA) and ethyl acrylate (EA), as polymer B Guar Hydroxypropyltrimonium Chloride and as polymer C VP/DMAPA Acrylates Copolymer (INCI).

Agents that are also particularly preferred contain as copolymer A a copolymer of methacrylic acid (MAA) and ethyl acrylate (EA), as polymer B Guar Hydroxypropyltrimonium Chloride and as polymer C Polyquaternium-11 (INCI).

Further agents that are likewise particularly preferred contain as copolymer A a copolymer of methacrylic acid (MAA) and ethyl acrylate (EA), as polymer B Guar Hydroxypropyltrimonium Chloride and as polymer C Polyquaternium-16 (INCI).

With regard to the selection of the (co)polymers A to C, the present disclosure is not subject to any limitations. Just one polymer or also a plurality of polymers from the individual described classes could be used.

It can be preferred in particular that the cosmetic agent contains two or more polymers C.

In this embodiment of the present disclosure agents contain as copolymer A a copolymer of methacrylic acid (MAA) and ethyl acrylate (EA), as polymer B Guar Hydroxypropyltrimonium Chloride and as polymer C vinylpyrrolidone-vinyl acetate copolymer (INCI: VP/VA Copolymer) and Polyquaternium-11 (INCI) are particularly preferred.

Alternative, particularly preferred agents contain as copolymer A a copolymer of methacrylic acid (MAA) and ethyl acrylate (EA), as polymer B Guar Hydroxypropyltrimonium Chloride and as polymer C vinylpyrrolidone-vinyl acetate copolymer (INCI: VP/VA Copolymer) and Polyquaternium-16 (INCI).

Regardless of the type and ratio by weight of the polymers to one another, agents as contemplated herein in which the total polymer content of (co)polymers A to C of the agent is from about 0.3 to about 40% by weight, preferably from about 1 to about 25% by weight, more preferably from about 4 to about 20% by weight and in particular from about 5 to about 15% by weight, are additionally preferred.

The cosmetic agent is provided/used in the form of a mousse, a foam or a spray with use of a propellant (for example aerosol spray).

Accordingly, a fourth essential ingredient of the agent for temporarily shaping keratin fibres is at least one propellant.

Suitable propellants (propellant gases) are propane, propene, n-butane, iso-butane, iso-butene, n-pentane, pentene, iso-pentane, iso-pentene, methane, ethane, dimethylether, nitrogen, air, oxygen, nitrous oxide, 1,1,1,3-tetrafluoroethane, heptafluoro-n-propane, perfluoroethane, monochlorodifluoromethane, 1,1-difluoroethane, more specifically either individually or in combination. Hydrophilic propellant gases such as carbon dioxide, can also be used advantageously in the context of the present disclosure, if the proportion of hydrophilic gases is selected to be low and a lipophilic propellant gas (for example propane/butane) is present in excess. Propane, n-butane, iso-butane, and mixtures of said propellant gases are particularly preferred. preferred cosmetic agents are exemplified in that the agent furthermore comprises at least one propellant b) from the group: propane, mixture of propane and butane, dimethyl ether and 1,1-difluoroethane (INCI: Hydrofluorocarbon 152a). A mixture of propane and butane is preferably used as propellant.

The amount of propellant is preferably from about 2 to about 15% by weight and more preferably from about 4 to about 12% by weight, in each case in relation to the total cosmetic agent.

The cosmetic agent can preferably be part of a cosmetic product which also comprises a dispensing device.

Vessels made of metal (aluminium, tinplate, tin), protected or non-splintering plastic, or glass that is externally coated with plastic may be used as compressed gas containers for aerosol applications; compressive strength and breaking strength, corrosion resistance, ease of filling, as well as aesthetic aspects, handling, printability, etc., play a role in their selection.

Special protective internal coatings ensure resistance to corrosion in relation to the cosmetic agent.

Should the cosmetic agents be sprayed onto the hair, these agents are advantageously dispensed in a dispensing device having a spray valve. The resultant cosmetic products accordingly comprise a cosmetic agent and a dispensing device with spray valve.

The agents contain the polymers and the propellant in a cosmetically acceptable carrier.

Preferred cosmetically acceptable carriers are aqueous, alcoholic or aqueous-alcoholic media with preferably at least about 10% by weight water, in relation to the total agent. In particular, the lower alcohols with 1 to 4 carbon atoms usually used for cosmetic purposes, for example ethanol and isopropanol, can be contained as alcohols. In a preferred embodiment the agent contains from about 3 to about 25% by weight and more preferably from about 5 to about 15% by weight, in each case in relation to the total agent, of ethanol.

In this embodiment of the present disclosure particularly preferred agents contain as copolymer A a copolymer of methacrylic acid (MAA) and ethyl acrylate (EA), as polymer B Guar Hydroxypropyltrimonium Chloride and as polymer C Polyquaternium-11 (INCI) and from about 5 to about 15% by weight, in relation to the total agent, of ethanol.

As additional co-solvents, organic solvents or a mixture of solvents with a boiling point below about 400° C. can be contained in an amount of from about 0.1 to about 15% by weight, preferably from about 1 to about 10% by weight, in relation to the total agent. Particularly suitable as additional co-solvents are unbranched or branched hydrocarbons, such as pentane, hexane, isopentane and cyclic hydrocarbons, such as cyclopentane and cyclohexane. Further particularly preferred water-soluble solvents are glycerol, ethylene glycol, butylene glycol, sorbitol and 1,2-propanediol in an amount up to about 30% by weight in relation to the total agent.

The agents preferably have a pH value of from about 2 to about 11. The pH range is particularly preferably between about 2 and about 8. The details regarding the pH value relate, in the context of this specification, to the pH value at 25° C., unless stated otherwise.

Further constituents of the agent for temporarily shaping keratin fibres will be described hereinafter and can be contained in the agents in addition to the mandatory ingredients described beforehand.

It can be preferred that the agent for temporarily shaping keratin fibres also comprises chitosan.

Chitosans are biopolymers and belong to the group of hydrocolloids. Considered chemically, they are partially deacetylated chitins of different molecular weight. In order to produce chitosans, chitin is used as a starting point, preferably the shell residues of crustaceans, which are available in large quantities as inexpensive raw materials. The chitin is usually first deprotonated here by addition of bases, is demineralised by addition of mineral acids, and lastly is deacetylated by addition of strong bases, wherein the molecular weights can be distributed over a broad spectrum. Types that have a mean molecular weight (weight average) of from about 800,000 to about 1,200,000 daltons, a Brookfield viscosity (about 1% by weight in glycolic acid) below about 5,000 mPas, a degree of deacetylation ranging from about 80 to about 88%, and an ash content of less than about 0.3 wt. % are preferably used.

Cationically derivatised chitosans (such as quaternisation products) or alkoxylated chitosans can also be used. Accordingly, the term “chitosan” includes chitosan or derivatives thereof.

Suitable chitosans are freely available commercially under the trade names Hydagen® CMF (about 1% by weight active substance in aqueous solution with about 0.4% by weight glycolic acid, molecular weight from about 500,000 to about 5,000,000 g/mol; Cognis), Hydamer® HCMF (Chitosan (deacetylated to about 80%), molecular weight from about 50,000 to about 1000000 g/mol, Chitinor, previously Cognis), Kytamer® PC (approximately 80% by weight active substance of chitosan pyrrolidone carboxylate (INCI name: Chitosan PCA), Amerchol), Chitolam® NB/101 and Chitosan 90/100/A1® (Chitosan (deacetylated to approximately 90%); BioLog Heppe).

The chitosan is contained in preferred agents, in relation to the total weight thereof, in a total amount of from about 0.01 to about 1.5% by weight, preferably from about 0.1 to about 1% by weight. The use of non-derivatised chitosan is particularly preferred.

Particularly preferred agents contain as copolymer A a copolymer of methacrylic acid (MAA) and ethyl acrylate (EA), as polymer B Guar Hydroxypropyltrimonium Chloride (INCI), as polymer C polyvinylpyrrolidone (PVP) and chitosan, preferably non-derivatised chitosan.

Agents that are likewise particularly preferred contain as copolymer A a copolymer of methacrylic acid (MAA) and ethyl acrylate (EA), as polymer B Guar Hydroxypropyltrimonium Chloride (INCI), as polymer C polyvinylpyrrolidone-vinyl acetate copolymer (INCI: VP/VA Copolymer) and Polyquaternium-16 (INCI) and chitosan, preferably non-derivatised chitosan.

It can be preferred that the agent for temporarily shaping keratin fibres also comprises nourishing substances for keratin fibres, in particular human hair, and/or human skin, in particular the scalp. These substances can comprise in particular D-panthenol, L-arginine, dimethicone (INCI), amodimethicone (INCI), vitamins, vitamin derivatives, protein hydrolysats, collagenhydrolysate and/or nicotinic acid amide.

As mentioned at the outset, the above-described cosmetic agents are exemplified by particular hair-cosmetic properties, in particular advantageous properties with regard to temporary hair shaping. A third subject of the present application is therefore the use of an agent or product as contemplated herein for the temporary shaping of keratin fibres, in particular human hair.

A fourth subject of the present application is a method for temporarily shaping keratin fibres, in particular human hair, in which the keratin fibres are exposed to a cosmetic agent as contemplated herein and are temporarily fixed in their form.

That which has been described with regard to the cosmetic agents applies, mutatis mutandis, with regard to further preferred embodiments of the use and the method.

### PRACTICAL EXAMPLES

The following amounts shall be understood to be percentages by weight of active substance, unless stated otherwise.

Products

The styling agents E1 to E6 as contemplated herein and the styling agents V1 to V4 not as contemplated herein were produced in accordance with the following Tables 1a and 1b. The ingredients are named in part in accordance with their INCI naming.

TABLE 1
Styling agent composition

| Raw materials | E1 | V1 | E2 | V2 | E3 |
|---|---|---|---|---|---|
| MAA/EA | 1 | — | 1 | — | 1 |
| Guar | 3 | — | 3 | — | 3 |
| Hydroxypropyltrimonium | | | | | |
| Chloride | | | | | |
| PVP (K-90) | 1 | 1 | — | — | — |
| PVP/VA (60/40) | — | — | 1 | 1 | 1 |
| Chitosan | 0.3 | 0.3 | — | 0.5 | 0.3 |
| Polyacrylamido- | 0.2 | 0.2 | | | |
| propyltrimonium | | | | | |
| Chloride | | | | | |
| Polyquaternium-4 | — | 1.35 | — | — | |
| Polyquaternium-11 | — | — | — | — | — |
| Polyquaternium-16 | — | — | 0.8 | 1.6 | 0.8 |
| Amodimethicone | — | — | 0.2 | 0.2 | 0.2 |
| Cetrimonium Chloride | 0.25 | 0.25 | 0.25 | 0.25 | 0.25 |
| Castor Oil | 0.2 | 0.2 | 0.2 | 0.2 | 0.2 |
| hydrog. 40 EO | | | | | |
| D-Panthenol | 0.1 | 0.1 | 0.1 | 0.1 | 0.1 |
| Lactic acid | 0.22 | 0.22 | 0.35 | 0.35 | 0.35 |
| Perfume | 0.2 | 0.2 | 0.2 | 0.2 | 0.2 |
| Preservatives | 0.5 | 0.3 | 0.5 | 0.3 | 0.3 |
| Propane/butane | 6 | 6 | 6 | 6 | 6 |
| Water, | to 100 | to 100 | to 100 | to 100 | to 100 |
| fully demineralised | | | | | |

TABLE 1
Styling agent composition

| Raw materials | V3 | E4 | E5 | E6 | V4 |
|---|---|---|---|---|---|
| MAA/EA | — | 0.5 | 0.5 | 1 | — |
| Guar | — | 1.5 | 1.5 | 3 | — |
| Hydroxypropyltrimonium | | | | | |
| Chloride | | | | | |
| PVP (K-90) | — | — | — | — | 0.2 |
| PVP/VA (60/40) | — | — | — | 2.5 | 5 |
| PVP/VA (70/30) | 3.2 | 1.6 | — | — | — |
| Chitosan | — | — | — | — | — |
| Polyacrylamido- | — | — | | 0.2 | 0.2 |
| propyltrimonium | | | | | |
| Chloride | | | | | |
| Polyquaternium-4 | — | — | — | — | 0.3 |
| Polyquatemium-11 | 2.2 | 1.1 | 1.1 | 0.75 | 1.5 |
| Polyquatemium-16 | — | — | — | — | — |
| Amodimethicone | — | — | — | 0.2 | — |
| Cetrimonium Chloride | 0.25 | 0.25 | 0.25 | 0.25 | 0.25 |
| Castor Oil | — | — | — | 0.2 | 0.2 |
| hydrog. 40 EO | | | | | |
| D-Panthenol | 0.1 | 0.1 | 0.1 | 0.1 | 0.1 |
| L-Arginine | — | — | — | 0.1 | 0.1 |
| Lactic acid | 0.26 | 0.26 | 0.26 | 0.14 | 0.14 |
| Perfume | 0.1 | 0.1 | 0.1 | 0.1 | 0.1 |
| Preservatives | 0.3 | 0.4 | 0.4 | 0.5 | 0.3 |
| Ethanol | 11 | 11 | 11 | — | — |
| 1,2-propanediol | — | — | — | 1.5 | 1.5 |
| Glycerol | — | — | — | 0.2 | 0.2 |
| Propane/Butane | 10 | 10 | 10 | 6 | 6 |
| Water, | to 100 | to 100 | to 100 | to 100 | to 100 |
| fully demineralised | | | | | |

The produced cosmetic agents were examined in respect of their moisture resistance by employing the HHCR test (High Humidity Curl Retention test).

To this end 10 hair strands (European natural, type 827560 attached at one end, not glued, colour 6/0, length 240 mm, weight ˜0.6 g, Lₘₐₓ=220 mm, Kerling Internationale Haarfarbrik GmbH) were prepared per cosmetic agent E1 to E6 and V1 to V4 by applying 180 mg of the cosmetic agent in question to the hair strands and massaging this in by hand. The hair strands were than wound on winders (length 160 mm, diameter 10 mm) and were dried overnight at 298 K and 50% relative humidity. Once the hair strands had been unwound they were secured to a metal frame, placed in a climatic chamber at 298 K and 85% relative humidity and their length was determined directly thereafter using a laser (corresponds to the value Lₒ). After a further 6 h the length of the hair strands was determined again *corresponds to value Lₜ).

The moisture resistance, i.e. the HHCR value, was calculated on the basis of the determined values in accordance with the following equation:

$$HHCR = \frac{{L}_{m ax} - {L}_{t}}{{L}_{m ax} - {L}_{0}} * 100$$

The HCCR values for the cosmetic agents E1 to E6 and V1 to V4 are given in the following table.

| | E1 | V1 | E2 | V2 | E3 | E4 | E5 | E6 | V3 | V4 |
|---|---|---|---|---|---|---|---|---|---|---|
| HHCR | 48 | 39 | 26 | 14 | 48 | 20 | 28 | 41 | 17 | 26 |
| [%] | | | | | | | | | | |

The HHCR value ascertained for cosmetic agent E1 is much higher than the HHCR value ascertained for the cosmetic agent V1. Due to the use of the combination of an anionic copolymer A based on methacrylic acid and ethyl acrylate with a cationically modified guar derivative B and PVP as polymer C, a significant increase in the moisture resistance of cosmetic agents for temporarily shaping keratin fibres is achieved as compared to a combination of a cationic cellulose derivative and polyvinylpyrrolidone.

The same is true for the cosmetic agents E2 and V2. Here as well, due to the use of the combination of an anionic copolymer A based on methacrylic acid and ethyl acrylate with a cationically modified guar derivative B instead of chitosan, a significant increase in the moisture resistance of cosmetic agents for temporarily shaping keratin fibres is achieved. In addition, it was possible to halve the amount of the synthetic polymer Polyquaternium-16.

A comparison of the HHCR values for the cosmetic agents E3 and V2 shows that, with the aid of a combination of an anionic copolymer A based on methacrylic acid and ethyl acrylate with a cationically modified guar derivative B, a significant increase in the moisture resistance of cosmetic agents for temporarily shaping keratin fibres can be achieved.

A comparison of the HHCR values for the cosmetic agents E4 and E5 with the HHCR value of the agent V3 also shows that, with the aid of a combination of an anionic copolymer A based on methacrylic acid and ethyl acrylate with a cationically modified guar derivative B with simultaneous reduction of the proportion of purely synthetic polymers (here: PVP/VA and Polyquaternium-11), an increase in the moisture resistance of cosmetic agents for temporarily shaping keratin fibres can be achieved.

The same is true for the comparison of the agents E6 and V4. The HHCR value of the agent E6, with simultaneous significant reduction of the proportion of purely synthetic polymers by the use of the combination of MAA/EA copolymer (=copolymer A) and cationic guar derivative B, is much higher than the HHCR value of the agent V4.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

What is claimed is:

1. A cosmetic agent for temporarily shaping keratin fibres, comprising in a cosmetically acceptable carrier:
a) at least one copolymer A formed from
a1) at least one monomer A1 selected from acrylic acid and/or methacrylic acid and
a2) at least one monomer A2 selected from acrylates and/or methacrylates;
b) at least one cationic polymer B different from copolymer A and selected from cationic guar derivatives;
c) at least one polymer C different from copolymer A and polymer B and the at least one polymer C is selected from homopolymers or copolymers of vinylpyrrolidone; and
d) at least one propellant.

2. The cosmetic agent for temporarily shaping keratin fibres according to claim 1, wherein the copolymer A comprises a copolymer of methacrylic acid (MAA) and ethyl acrylate (EA).

3. The cosmetic agent for temporarily shaping keratin fibres according to claim 1, wherein the cationic guar derivatives is guar 2-hydroxy-3-(trimethylammonio)propyl ether (INCI name: Guar Hydroxypropyltrimonium Chloride).

4. The cosmetic agent for temporarily shaping keratin fibres according to claim 1, wherein the polymer C is selected from the group of polyvinylpyrrolidone (PVP), vinylpyrrolidone-vinyl acetate copolymer (VP/VA Copolymer), Vinyl Caprolactam/VP/Dimethylaminoethyl Methacrylate Copolymer (INCI), VP/DMAPA Acrylates Copolymer (INCI), Polyquaternium-11 (INCI), Polyquaternium-16 (INCI), and combinations thereof.

5. The cosmetic agent for temporarily shaping keratin fibres according to claim 1, further comprising chitosan.

6. The cosmetic agent for temporarily shaping keratin fibres according to claim 1, wherein a ratio by weight of copolymer A to polymer(s) B is from about 1:1 to about 1:10.

7. The cosmetic agent for temporarily shaping keratin fibres according to claim 1, further comprising from about 3 to about 25% by weight, in relation to the total agent, of ethanol.

8. A cosmetic product comprising:
i. an agent for temporarily shaping keratin fibres according to claim 1, and
ii. a dispensing device.

9. A method for temporarily shaping keratin fibres comprising:
exposing the keratin fibres to a cosmetic agent according to claim 1; and
temporarily fixing the keratin fibres in form.

10. The cosmetic agent for temporarily shaping keratin fibres according to claim 1, wherein a ratio by weight of copolymer A to polymer(s) B is from about 1:1 to about 1:5.

11. The cosmetic agent for temporarily shaping keratin fibres according to claim 1, wherein a ratio by weight of copolymer A to polymer(s) B is from about 1:2 to about 1:4.

12. The cosmetic agent for temporarily shaping keratin fibres according to claim 1, further comprising from about 5 to about 15% by weight, in relation to the total agent, of ethanol.

13. The cosmetic product according to claim 8, wherein the dispensing device is a spray valve.

14. The method according to claim 9 wherein the keratin fibres is human hair.

15. A cosmetic agent for temporarily shaping keratin fibres comprising:
from about 0.25 to about 1.5% by weight, in relation to the total weight of the cosmetic agent, of a copolymer of methacrylic acid and ethyl acrylate;
from about 0.75 to about 4.5% by weight, in relation to the total weight of the cosmetic agent, of guar 2-hydroxy-3-(trimethylammonio)propyl ether;
from about 1 to about 10% by weight, in relation to the total weight of the cosmetic agent, of homopolymers or copolymers of vinylpyrrolidone selected from polyvinylpyrrolidone (PVP), vinylpyrrolidone-vinyl acetate copolymer (PVP/VA), Polyquaternium-11 (INCI), Polyquaternium-16 (INCI), or combinations thereof; and
from about 4 to about 12% by weight, in relation to the total weight of the cosmetic agent, of a propellant selected from propane, butane, and combinations thereof.
